(19) United States
(12) Patent Application Publication
(10) Pub. No.: US 2025/0002960 A1
(43) Pub. Date: Jan. 2, 2025

(54) COMPOSITIONS AND METHODS FOR NUCLEIC ACID EXTENSION

(71) Applicant: Pacific Biosciences of California, Inc., Menlo Park, CA (US)

(72) Inventors: Sima YAZDANI, Menlo Park, CA (US); Chih-Yuan CHEN, Menlo Park, CA (US); Richard LECOULTRE, Menlo Park, CA (US); Lubomir SEBO, Menlo Park, CA (US); Joshua GARRETSON, Menlo Park, CA (US)

(73) Assignee: Pacific Biosciences of California, Inc., Menlo Park, CA (US)

(21) Appl. No.: 18/753,625

(22) Filed: Jun. 25, 2024

Related U.S. Application Data
(60) Provisional application No. 63/523,257, filed on Jun. 26, 2023.

(51) Int. Cl. C12P 19/34

(52) U.S. Cl. CPC C12P 19/34

(57) ABSTRACT

The present disclosure relates to a deblocking agent and compositions thereof for cleaving an NO bond in an amineoxy group at the 3′ position of a non-extendable polynucleotide, the deblocking agent being a carbonyl phosphonate and/or sulfonate, or salt thereof. The present disclosure further relates to methods of activating and/or extending a non-extendable polynucleotide using the deblocking agent of the present disclosure. The present disclosure also relates to methods of sequencing a target nucleic acid comprising the methods of activating a non-extendable polynucleotide of the present disclosure.

### CROSS-REFERENCE TO RELATED APPLICATIONS

This application is based on, and claims the benefit of, U.S. Provisional Application No. 63/523,257, filed Jun. 26, 2023, the which is incorporated herein by reference in its entirety.

### FIELD

The present disclosure refers to a method of activating a non-extendable polynucleotide, the non-extendable polynucleotide having an amineoxy group. The method comprises contacting the polynucleotide with a deblocking agent to cleave a NO bond of the amineoxy group to obtain an extendable polynucleotide and extending the extendable polynucleotide. The present disclosure also relates to a deblocking composition comprising the deblocking agent of the present disclosure, a buffer and a solubility enhancer. The present disclosure further relates to methods of polymerizing polynucleotides in sequencing or synthesis reactions.

### INTRODUCTION

In a number of nucleic acid related chemical manipulations, it may be desirable to reversibly terminate extension of a polynucleotide chain, for example to allow for detection, characterization, isolation, further modification, etc. Since extension of a polynucleotide chain usually requires attachment of the next nucleotide to the 3′-OH group of the growing polynucleotide chain, termination of extension can be achieved by blocking or masking of the 3′-OH group. To this effect, amineoxy group (—ONR₂, such as ONH₂, also referred to as an aminooxy group) has been used to replace 3′-OH group of polynucleotide to prevent addition of the incoming nucleotide. See for example U.S. Ser. No. 10/472,382.

When the amineoxy group is exposed to certain reagents, the nitrogen-oxygen (NO) bond of the amineoxy group can be cleaved to reveal the 3′-OH group for further extension. This reversible blocking of extension is particularly useful in sequencing by synthesis applications, where detection can occur after the incorporation of each non-extendable polynucleotide before the non-extendable polynucleotide is deblocked to allow for further extension.

A number of reagents have been suggested for NO bond cleavage. U.S. Pat. No. 8,034,923 uses sodium nitrite as a deblocking agent to cleave 3′-ONH₂ group. U.S. Pat. No. 7,544,794 describes a number of reagents that have been suggested in the literature that might be used to cleave NO bonds, and states nitrous acid (HNO₂) at pH 4-5 is a preferred cleavage method. As such solution of NO₂ salt, such as NaNO₂ or a NaOAc salt, at low pH (e.g, pH 4-5) can provide nitrous acid suitable for cleaving an NO bond. However, the reagents used or suggested are often harsh or require harsh reaction conditions (high temperature, high acidity, etc.) that would damage single strand DNA (e.g. template polynucleotide) or are costly. Other cleavage reagents were designed for simple and/or specific molecular structures comprising NO bonds and may not be compatible with nucleic acid.

Accordingly, there exists a need to develop reagents that can achieve mild cleavage of NO bond at the 3′ position of a polynucleotide.

### SUMMARY

It has been shown that carbonyl phosphonate and carbonyl sulfonate compounds can be used as deblocking agents to cleave NO bond in an amineoxy group at the 3′ position of a polynucleotide without any single strand nucleic acid damage. It has also been shown that solubility enhancers such as amines can be used with the carbonyl phosphonate or carbonyl sulfonate deblocking agent to facilitate the cleavage reaction by minimizing precipitation of the deblocking agent.

Accordingly, in one aspect, the present disclosure includes a method of activating a non-extendable polynucleotide comprising
contacting the non-extendable polynucleotide with a deblocking agent, the non-extendable polynucleotide having an amineoxy group at the 3′ position, to cleave an NO bond of the amineoxy group to obtain an extendable polynucleotide, and
extending the extendable polynucleotide in a nucleic acid polymerisation,
wherein the deblocking agent is a carbonyl phosphonate and/or a carbonyl sulfonate.

In another aspect, the present disclosure includes a use of a deblocking agent in activating a non-extendable polynucleotide for polymerization, wherein the non-extendable polynucleotide has an amineoxy group at the 3′ position, and wherein the deblocking agent is as defined in the present disclosure.

In another aspect, the present disclosure includes a deblocking composition comprising
a deblocking agent as defined herein,
a buffer, and
a solubility enhancer.

In another aspect, the present disclosure includes a kit comprising the deblocking composition of the present disclosure and instructions for use.

### FIGURES

The embodiments of the disclosure will now be described in greater detail with reference to the attached drawings in which:

FIG. 1 shows electropherograms of Octet/CE cyclic extension assay design (Panel A), results using 37 cycles of extension with NaNO₂ (Panel B) and degradation of the extended polynucleotide after 24 hours of incubation of with NaNO₂ (Panel C).

FIG. 2 shows electropherograms of Octet/CE cyclic extension assays using 37 cycles of extension with either NaNO₂ (Panel A), CDP alone (Panel B) or CDP and N,N′-Dimethylethylenediamine in sodium acetate buffer (Panel C) as deblocking composition.

FIG. 3 shows raw intensities of on/off sequencing run. Y axis is on/off intensities. Each solid line of different color is signal (ON) from a different base, and each dotted line is background for that signal (OFF) for a different base over 150 cycles.

FIG. 4 shows electropherograms of Octet/CE runoff assay design (Panel A) and results (Panel B) showing reduction in ssDNA damage by CDP compared to NaNO₂.

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments, but should be given the broadest interpretation consistent with the description as a whole.

### DESCRIPTION OF VARIOUS EMBODIMENTS

### I. Definitions

Unless otherwise indicated, the definitions and embodiments described in this and other sections are intended to be applicable to all embodiments and aspects of the present disclosure herein described for which they are suitable as would be understood by a person skilled in the art.

The term “deblocking agent of the disclosure” or “deblocking agent of the present disclosure” and the like as used herein refers to a carbonyl phosphonate or carbonyl sulfonate compound, or salts thereof. For example, the deblocking agent can be a compound of Formula I or a salt thereof.

The term “deblocking composition(s) of the disclosure” or “deblocking composition(s) of the present disclosure” and the like as used herein refers to a composition comprising one or more deblocking agents of the disclosure.

The term “and/or” as used herein means that the listed items are present, or used, individually or in combination. In effect, this term means that “at least one of” or “one or more” of the listed items is used or present.

As used in the present disclosure, the singular forms “a”, “an” and “the” include plural references unless the content clearly dictates otherwise. For example, an embodiment including “a compound” should be understood to present certain aspects with one compound, or two or more additional compounds.

In embodiments comprising an “additional” or “second” component, such as an additional or second compound, the second component as used herein is chemically different from the other components or first component. A “third” component is different from the other, first, and second components, and further enumerated or “additional” components are similarly different.

As used in this disclosure and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “consisting” and its derivatives as used herein are intended to be closed terms that specify the presence of the stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The term “suitable” as used herein means that the selection of the particular compound or conditions would depend on the specific synthetic manipulation to be performed, the identity of the molecule(s) to be transformed and/or the specific use for the compound, but the selection would be well within the skill of a person trained in the art.

In embodiments of the present disclosure, the compounds described herein may have at least one asymmetric center. Where compounds possess more than one asymmetric center, they may exist as diastereomers. It is to be understood that all such isomers and mixtures thereof in any proportion are encompassed within the scope of the present disclosure. It is to be further understood that while the stereochemistry of the compounds may be as shown in any given compound listed herein, such compounds may also contain certain amounts (for example, less than 20%, suitably less than 10%, more suitably less than 5%) of compounds of the present disclosure having an alternate stereochemistry. It is intended that any optical isomers, as separated, pure or partially purified optical isomers or racemic mixtures thereof are included within the scope of the present disclosure.

The compounds of the present disclosure may also exist in different tautomeric forms and it is intended that any tautomeric forms which the compounds form, as well as mixtures thereof, are included within the scope of the present disclosure.

The present description refers to a number of chemical terms and abbreviations used by those skilled in the art. Nevertheless, definitions of selected terms are provided for clarity and consistency.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

The term “alkyl” as used herein, whether it is used alone or as part of another group, means straight or branched chain, saturated alkyl groups. The number of carbon atoms that are possible in the referenced alkyl group are indicated by the prefix “Cₙ₁₋ₙ₂”. For example, the term C₁₋₁₀alkyl means an alkyl group having 1, 2, 3, 4, 5, 6, 7, 8, 9 or 10 carbon atoms.

The term “alkylene”, whether it is used alone or as part of another group, means straight or branched chain, saturated alkylene group, that is, a saturated carbon chain that contains substituents on two of its ends. The number of carbon atoms that are possible in the referenced alkylene group are indicated by the prefix “Cₙ₁₋ₙ₂”. For example, the term C₂₋₆alkylene means an alkylene group having 2, 3, 4, 5 or 6 carbon atoms.

The term “alkenyl” as used herein, whether it is used alone or as part of another group, means straight or branched chain, unsaturated alkyl groups containing at least one double bond. The number of carbon atoms that are possible in the referenced alkyl group are indicated by the prefix “Cₙ₁₋ₙ₂”. For example, the term C₂₋₆alkenyl means an alkenyl group having 2, 3, 4, 5 or 6 carbon atoms.

The term “alkylene” as used herein, whether it is used alone or as part of another group, means a straight or branched chain, saturated alkylene group, that is, a saturated carbon chain that contains substituents on two of its ends. The number of carbon atoms that are possible in the referenced alkylene group are indicated by the prefix “Cₙ₁₋ₙ₂”. For example, the term C₁₋₆alkylene means an alkylene group having 1, 2, 3, 4, 5 or 6 carbon atoms. All alkylene groups are optionally fluorosubstituted unless otherwise stated.

The term “cycloalkyl,” as used herein, whether it is used alone or as part of another group, means a saturated carbocyclic group containing one or more rings. The number of carbon atoms that are possible in the referenced cycloalkyl group are indicated by the numerical prefix “Cₙ₁₋ₙ₂”. For example, the term C₃₋₁₀cycloalkyl means a cycloalkyl group having 3, 4, 5, 6, 7, 8, 9 or 10 carbon atoms.

The term “aryl” as used herein, whether it is used alone or as part of another group, refers to carbocyclic groups containing at least one aromatic ring. In an embodiment of the application, the aryl group contains from 6, 9 or 10 carbon atoms, such as phenyl, indanyl or naphthyl.

The term “heterocycloalkyl” as used herein, whether it is used alone or as part of another group, refers to cyclic groups containing at least one non-aromatic ring in which one or more of the atoms are a heteroatom selected from O, S and N. Heterocycloalkyl groups are either saturated or unsaturated (i.e. contain one or more double bonds). When a heterocycloalkyl group contains the prefix Cₙ₁₋ₙ₂ this prefix indicates the number of carbon atoms in the corresponding carbocyclic group, in which one or more, suitably 1 to 5, of the ring atoms is replaced with a heteroatom as defined above.

The term “heteroaryl” as used herein, whether it is used alone or as part of another group, refers to cyclic groups containing at least one heteroaromatic ring in which one or more of the atoms are a heteroatom selected from O, S and N. When a heteroaryl group contains the prefix Cₙ₁₋ₙ₂ this prefix indicates the number of carbon atoms in the corresponding carbocyclic group, in which one or more, suitably 1 to 5, of the ring atoms is replaced with a heteroatom as defined above.

All cyclic groups, including aryl, heteroaryl, cycloalkyl and heterocycloalkyl groups, contain one or more than one ring (i.e. are polycyclic). When a cyclic group contains more than one ring, the rings may be fused, bridged, spirofused or linked by a bond.

A first ring being “fused” with a second ring means the first ring and the second ring share two adjacent atoms there between.

A first ring being “bridged” with a second ring means the first ring and the second ring share two non-adjacent atoms there between.

A first ring being “spirofused” with a second ring means the first ring and the second ring share one atom there between.

The term “halo” as used herein refers to a halogen atom and includes fluoro, chloro, bromo and iodo.

The term “optionally substituted” refers to groups, structures, or molecules that are either unsubstituted or are substituted with one or more substituents.

The term “fluorosubstituted” refers to the substitution of one or more, including all, hydrogens in a referenced group with fluorine.

The symbol “ [symbol] ” is used herein to represent the point of attachment of a group to the remainder of a molecule or chemical formula.

The symbol “ [symbol] ” is used herein to represent an optional double bond. For example, the symbol “ [symbol] ” can be a single or a double bond.

The term “available”, as in “available hydrogen atoms” or “available atoms” refers to atoms that would be known to a person skilled in the art to be capable of replacement by a substituent.

The term “amine” or “amino,” as used herein, whether it is used alone or as part of another group, refers to groups of the general formula NR′R″, wherein R′ and R″ are each independently selected from hydrogen and C₁₋₂₀alkyl.

As used herein, “nucleic acid” or “oligonucleotide” or “polynucleotide” means at least two nucleotides covalently linked together. Thus, the terms include, but are not limited to, DNA, RNA, analogs (e.g., derivatives) thereof or any combination thereof, that can be acted upon by a polymerizing enzyme during nucleic acid synthesis. The term includes single-, double-, or multiple-stranded DNA, RNA and analogs (e.g., derivatives) thereof. Double-stranded nucleic acids advantageously can minimize secondary structures that may hinder nucleic acid synthesis. A double stranded nucleic acid may possess a nick or a single-stranded gap. A nucleic acid may represent a single, plural, or clonally amplified population of nucleic acid molecules. The polynucleotide may be a primer hybridized to a template nucleic acid.

As used herein, a “template nucleic acid” is a nucleic acid to be detected, sequenced, evaluated or otherwise analyzed using a method or apparatus disclosed herein.

As used herein, a “primed template nucleic acid” (or alternatively, “primed template nucleic acid molecule”) is a template nucleic acid primed with (i.e., hybridized to) a primer, wherein the primer is an oligonucleotide having a 3′-end with a sequence complementary to a portion of the template nucleic acid. The primer can optionally have a free 5′-end (e.g., the primer being noncovalently associated with the template) or the primer can be continuous with the template (e.g., via a hairpin structure). The primed template nucleic acid includes the complementary primer and the template nucleic acid to which it is bound. Unless explicitly stated, a primed template nucleic acid can have either a 3′ end that is extendible by a polymerase, or a 3′ end that is blocked from extension. An “extendable primed template nucleic acid molecule” is extendable in a polymerization reaction.

As used herein, a “blocked primed template nucleic acid” (or alternatively, “blocked primed template nucleic acid molecule”) is a primed template nucleic acid modified to preclude or prevent phosphodiester bond formation at the 3′-end of the primer. Blocking may be accomplished, for example, by chemical modification with a blocking group at either the 3′ or 2′ position of the five-carbon sugar at the 3′ terminus of the primer. Alternatively, or in addition, chemical modifications that preclude or prevent phosphodiester bond formation may also be made to the nitrogenous base of a nucleotide. Reversible terminator nucleotide analogs including each of these types of blocking groups will be familiar to those having an ordinary level of skill in the art. Incorporation of these analogs at the 3′ terminus of a primer of a primed template nucleic acid molecule results in a blocked primed template nucleic acid molecule. The blocked primed template nucleic acid includes the complementary primer, blocked from extension at its 3′-end, and the template nucleic acid to which it is bound.

The term “polymerase” as used herein refers to any protein, or complex thereof, that catalyzes the addition of a nucleotide to the 3′-oxygen of a polynucleotide via a phosphodiester bond, thereby chemically incorporating the nucleotide into the polynucleotide. The polynucleotide may be a primer hybridized to a template for template-based polymerization, or in the case of template independent polymerization (e.g., ssDNA synthesis by TdT) may not be hybridized to a template. Polymerase may form a ternary complex with a cognate nucleotide and primed template nucleic acid (or blocked primed template nucleic acid) including but not limited to, DNA polymerase, RNA polymerase, reverse transcriptase, primase and transferase. Typically, the polymerase includes one or more active sites at which nucleotide binding may occur. Optionally a polymerase includes one or more active sites at which catalysis of nucleotide polymerization may occur. Optionally a polymerase lacks catalytic nucleotide polymerization function, for example, due to a modification such as a mutation or chemical modification. Alternatively, the polymerase may catalyze the polymerization of nucleotides to the 3′-end of a primer bound to its complementary nucleic acid strand. Optionally, the polymerase used in the provided methods is a processive polymerase. Optionally, the polymerase used in the provided methods is a distributive polymerase.

### II. Deblocking Agents and Compositions of the Disclosure

In one aspect, the present disclosure includes a deblocking agent that is a carbonyl phosphonate and/or a carbonyl sulfonate or salt thereof. A phosphonate may comprise a C—PO(OH)₂ group. A sulfonate may comprise a C—SO₂OH group. Deblocking may be referred to as cleaving or deprotecting.

In some embodiments, the deblocking agent is comprised in a deblocking composition, the deblocking composition comprising the deblocking agent and a buffer. In some embodiments, the deblocking solution further comprises a solubility enhancer.

In another aspect, the present disclosure includes a deblocking composition comprising:
a deblocking agent as defined herein,
a buffer, and
a solubility enhancer.

In another aspect, the present disclosure includes a kit comprising the deblocking composition of the present disclosure and instructions for use.

In some embodiments, the deblocking composition has a pH of about 4 to about 8. In some embodiments, the deblocking composition has a pH of about 4 to about 7. In some embodiments, the deblocking composition has a pH of about 4 to about 6, about 4 to about 5.5, about 4.5 to about 6, or about 5 to about 5.7.

In some embodiments, the buffer comprises or is acetate.

In some embodiments, the solubility enhancer is an amine. In some embodiments, the amine is selected from substituted or unsubstituted nitrogen-containing heteroaromatic, substituted or unsubstituted arylamine, alkylamine, substituted or unsubstituted nitrogen-containing heterocycle, and combinations thereof.

In some embodiments, the amine is selected from N,N′-dialkylalkylenediamine, substituted or unsubstituted aniline, substituted or unsubstituted imidazole, substituted or unsubstituted benzimidazole, substituted or unsubstituted pyridine, substituted or unsubstituted trialkylamine, DABCO, DBU, and combination thereof.

In some embodiments, the amine is selected from N,N′-dimethylethylenediamine, N,N′-diethylethylenediamine, 1-methylimidazole, imidazole, 2-amino-5-methoxybenzoic acid, 2-(aminomethyl)benzimidazole, benzimidazole, trimethylamine, troethylamine, DABCO, DBU, and combinations thereof. In some embodiments, the amine is N,N′-dimethylethylenediamine.

In some embodiments, the solubility enhancer is present in the deblocking composition at a concentration of at least 1 mM, or of about 1 mM to about 500 mM, about 10 mM to about 50 mM, about 20 mM to about 40 mM, about 25 mM to about 35 mM, or about 30 mM.

In some embodiments, the solubility enhancer acts as a counterion of the deblocking agent. For example, the solubility enhancer may be an amine counterion to the deblocking agent. Without wishing to be bound by theory, the solubility enhancer when acting as a counterion for the carbonyl phosphonate or the carbonyl sulfonate deblocking agent can help stabilize the carbonyl phosphonate or the carbonyl sulfonate in solution such that the carbonyl phosphonate or the carbonyl sulfonate does not precipitate out of solution.

In some embodiments, the deblocking agent is present in the deblocking composition at a concentration of at least 1 mM, or of about 1 mM to about 1 M, about 10 mM to about 100 mM, about 20 mM to about 80 mM, about 20 mM to about 70 mM, about 20 mM to about 60 mM, about 20 mM to about 50 mM, about 20 mM to about 40 mM, or about 30 mM.

Without wishing to be bound by theory, the mechanism of cleavage of NO bond by carbonyl phosphonate compound is postulated to involve as a first step the formation of oxime between the amineoxy (—ONH₂) and the carbonyl group of the deblocking agent. A rearrangement of the oxime with the phosphonate group leads to the cleavage of NO bond and the formation of H₃PO₄ as a byproduct. The mechanism for carbonyl diphosphonic acid (CDP) and O-alkylhydroxylamine as proposed by Khomich et al., Molecules, 2017, 22, 1040, the content of which is incorporated by reference. In general, the proposed cleavage mechanism for carbonyl phosphonate compound and R-hydroxylamine is shown in Scheme 1.

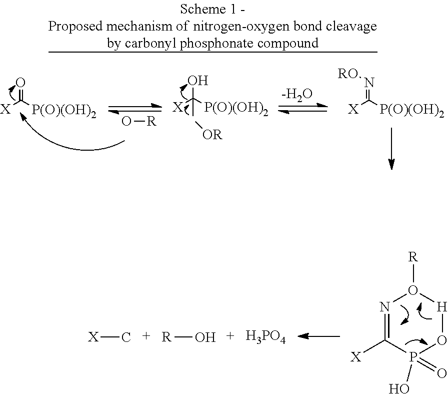

As suggested in the proposed mechanism, the carbonyl group forming the oxime should be in proximity of the phosphonate or sulfonate group. As such, it is contemplated that the deblocking agent of the present disclosure can have 0, 1, or 2 atoms between the carbonyl group and the phosphonate or sulfonate group. In some embodiments, the deblocking agent is a α-carbonyl phosphonate and/or a α-carbonyl sulfonate, or a salt thereof. In some embodiments, the deblocking agent is a β-carbonyl phosphonate and/or a β-carbonyl sulfonate, or a salt thereof. In some embodiments, a carbonyl group of the deblocking agent is adjacent to a phosphonate or sulfonate group of the deblocking agent.

It is also contemplated that the deblocking agent comprises a carbonyl group that is suitable for forming an oxime with the amineoxy group of the non-extendable polynucleotide, and at least one phosphonate or sulfonate group. In some embodiments, the carbonyl group of the deblocking agent is an aldehyde or a ketone. In some embodiments, the deblocking agent is a carbonyl diphosphonate, a carbonyl disulfonate, or comprises both a sulfonate group and a phosphonate group. In some embodiments, the deblocking agent only comprises a single carbon.

It can be appreciated that the deblocking agent can also comprise a carbonyl group and a group that can be hydrolysed to reveal a phosphonate or sulfonate group. In certain aspects, one or more phosphonate groups of a deblocking reagent described herein may be substituted with a group that can be hydrolysed to reveal a phosphonate. In certain aspects, one or more sulfonate groups of a deblocking reagent described herein may be substituted with a group that can be hydrolysed to reveal a sulfonate. For example, the deblocking agent can comprise a carbonyl and a sulfone group. For example, the deblocking agent can comprise a carbonyl and a phosphine oxide group, or a carbonyl and a phosphinic acid group.

In some embodiments, the deblocking agent of the present disclosure is compound of the structure of Formula I

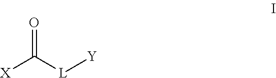

or a salt thereof,
wherein
Y is selected from

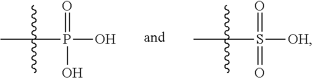 [SMILES: *P(=O)(O)O ; *S(=O)(=O)O]

L is absent or is selected from

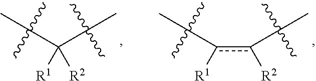

and substituted or unsubstituted

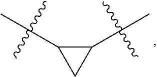 [SMILES: *C1CC1*]

wherein a phosphonate is a compound with a C—PO(OH)₂ group, and wherein a sulfonate is a compound with a C—SO(OH)₂ group,
   wherein R¹ and R² are each independently selected from H, C₁₋₂₀alkyl, C₃₋₂₀cycloalkyl, aryl, benzyl and combinations thereof, or
   wherein R¹ and R² together with the carbon atom(s) they are attached to are joined together to form a substituted or unsubstituted 3- to 8-membered cyclic structure,
X is selected from L-Y and R³, wherein R³ is selected from H, C₁₋₂₀alkyl, aryl, heteroaryl, C₂₋₂₀alkenyl, C₁₋₈alkylenearyl, C₃₋₂₀cycloalkyl, and combinations thereof, or X comprises a hydrophilic polymer, and
wherein each occurrence of alkyl, cycloalkyl, aryl, alkylenearyl, benzyl, and alkenyl is independently unsubstituted or substituted.

R¹ and R² are each independently selected from H, C₁₋₈alkyl, C₃₋₈cycloalkyl, aryl, benzyl and combinations thereof.

In some embodiments, R³ is selected from H, C₁₋₈alkyl, aryl, heteroaryl, C₂₋₈alkenyl, C₁₋₈alkylenearyl, C₃₋₈cycloalkyl.

In some embodiments, L is absent.

In some embodiments, X is L-Y. In some embodiments, X is Y.

In some embodiments, R¹ and R² are each independently selected from H, methyl, ethyl, OMe, OEt, pheyl, and combinations thereof.

In some embodiments, L is

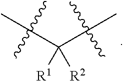

In some embodiments, R¹ and R² together with the carbon atom they are attached to are joined together to form the substituted or unsubstituted 3- to 8-membered cyclic structure. In some embodiments, the substituted or unsubstituted 3- to 8-membered cyclic structure is selected from C₃₋₆ cycloalkyl, and 3- to 6-membered heterocyclyl. In some embodiments, the substituted or unsubstituted 3- to 8-membered cyclic structure is selected from C₅₋₆ cycloalkyl, and 5- to 6-membered heterocyclyl.

In some embodiments, L is

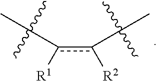

In some embodiments, R¹ and R² together with the carbon atoms they are attached to are joined together to form the substituted or unsubstituted 3- to 8-membered cyclic structure.

In some embodiments, the substituted or unsubstituted 3- to 8-membered cyclic structure is selected from C₃₋₆ cycloalkyl, 3- to 6-membered heterocyclyl, aryl, and 5-membered to 6-membered heteroaryl.

In some embodiments, R¹ and R² together with the carbon atom(s) they are attached to are joined together to form a 5- to 8-membered cyclic structure, optionally wherein the 5- to 8-membered cyclic structure is substituted with a

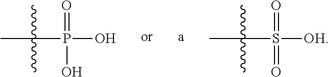 [SMILES: *P(=O)(O)O ; *S(=O)(=O)O]

In some embodiments, X is R³. In some embodiments, R³ is selected from C₁₋₃alkyl, phenyl, benzyl, imidazolyl, pyridinyl, C₃₋₆cycloalkyl, and combinations thereof.

In some embodiments, R³ is substituted with one or more polar substituents, optionally each independently selected from ester, hydroxyl, amine, OC₁₋₃alkyl, SH.

In some embodiments, R³ is substituted with one or more electron withdrawing groups (EWG), optionally each independently selected from halo, nitro, amide, ester, sulfonamide, and —C(O)C₁₋₃.

In some embodiments, X comprises a hydrophilic polymer. In some embodiments, the hydrophilic polymer is selected from polyethyleneglycol (PEG), polyvinylalcohol (PVA), and combinations thereof.

In some embodiments, the deblocking agent is selected from

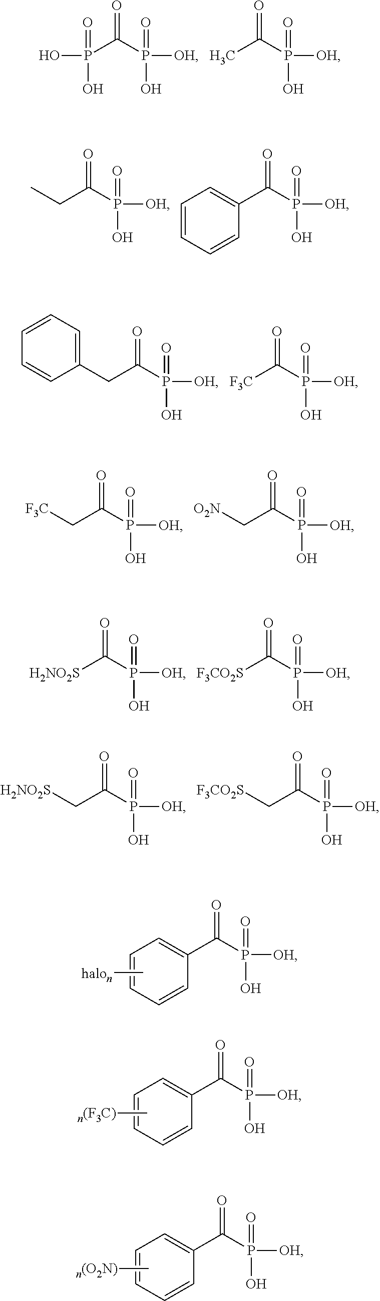

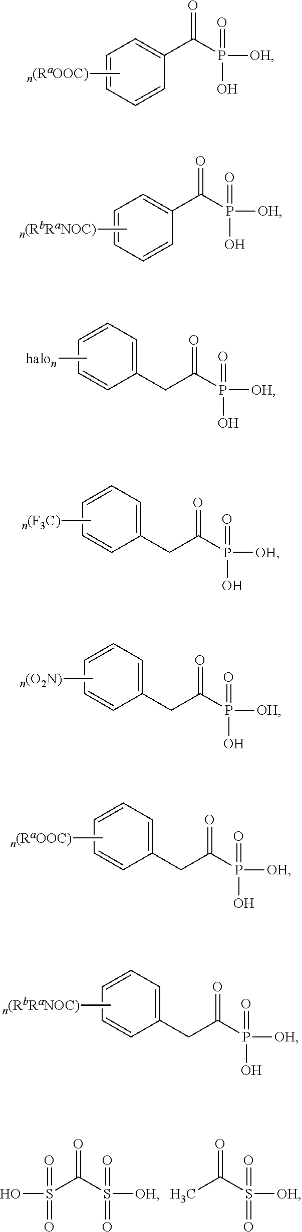

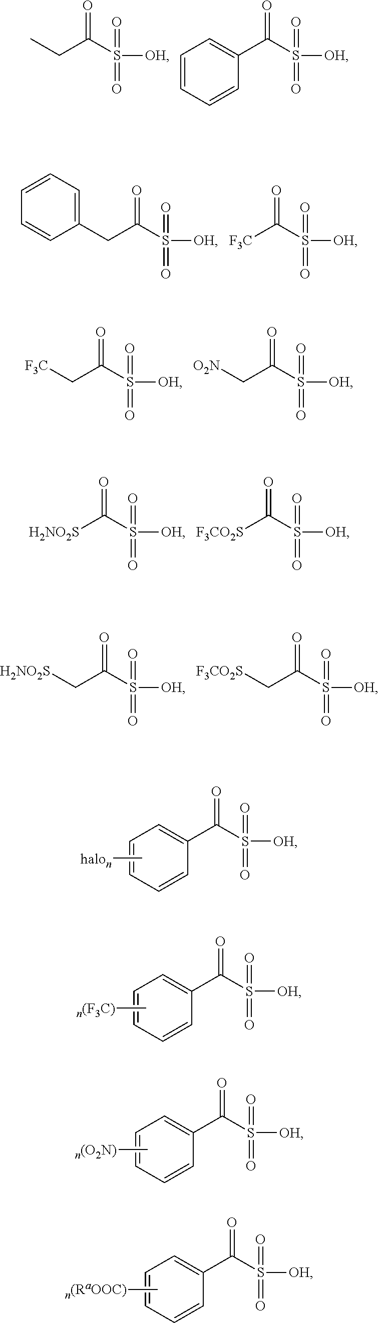

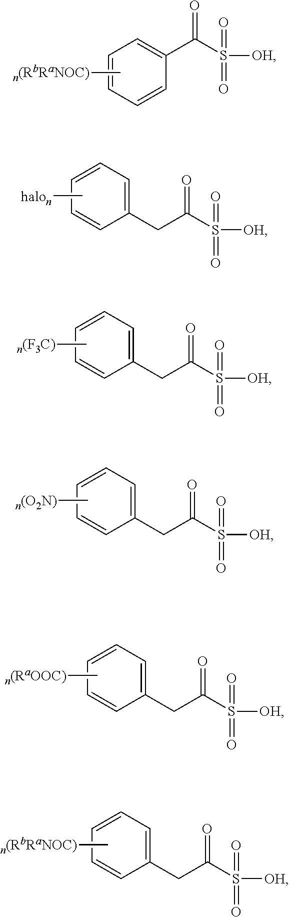

and salts thereof, wherein each halo is selected from F, Br, Cl, and I, each n is independently an integer of 1 to 3, and each occurrence of R^{a} and R^{b} is independently C₁₋₃alkyl.

In some embodiments, the deblocking agent is selected form

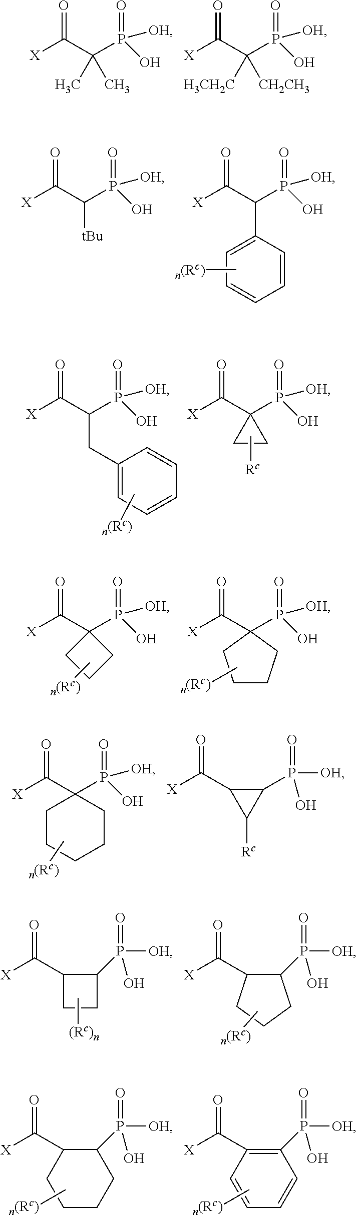

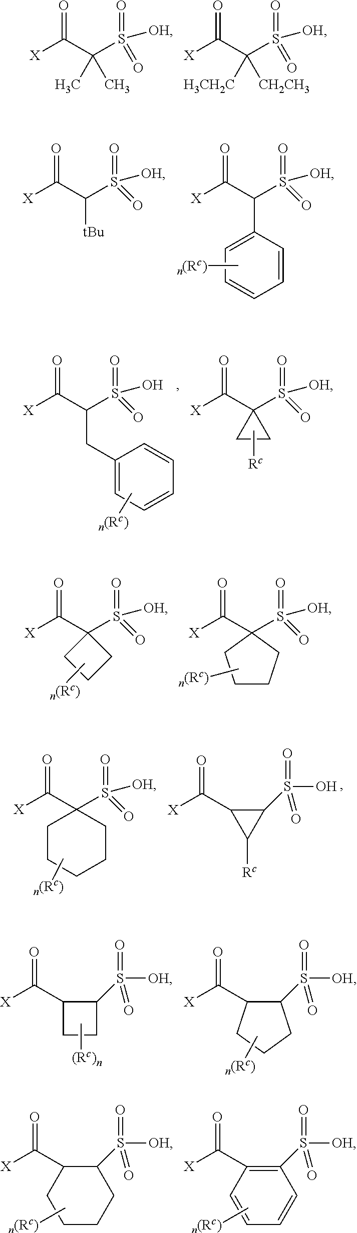

and salts thereof, wherein each occurrence of R^{c} is independently selected from H, C₁₋₃ alkyl,

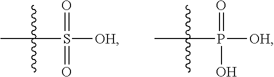 [SMILES: *S(=O)(=O)O ; *P(=O)(O)O]

and combinations thereof, and each occurrence of n is independently an integer of 0 to 3.

In some embodiments, X is selected from H, methyl, ethyl, phenyl, benzyl, —CF₃, —CH₂CF₃, —CH₂NO₂. —SO₂NH₂, —SO₂CF₃, —CH₂SO₂NH₂, —CH₂SO₂CF₃,

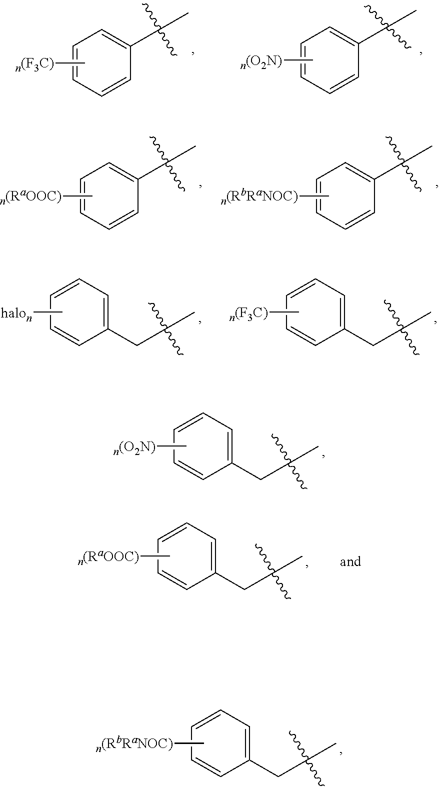

wherein R^{a}, R^{b}, n and halo are each as defined herein.

In some embodiments, the deblocking agent is selected from

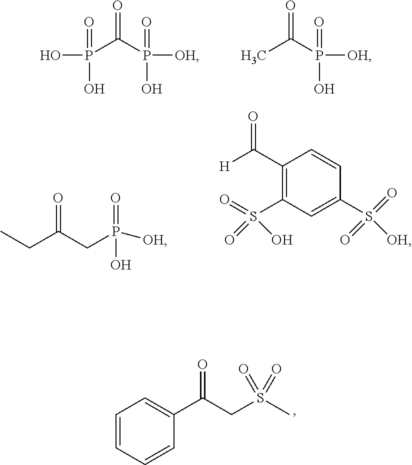

salts thereof, and combinations thereof.

In some embodiments, the deblocking agent is

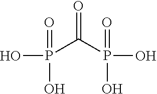

or a salt thereof.

In an embodiment, the salt is an acid addition salt or a base addition salt.

An acid addition salt is organic or inorganic acid addition salt of any basic compound. Basic compounds that form an acid addition salt include, for example, compounds comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di- and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. In an embodiment, the mono- or di-acid salts are formed, and such salts exist in either a hydrated, solvated or substantially anhydrous form. In general, acid addition salts are more soluble in water and various hydrophilic organic solvents, and generally demonstrate higher melting points in comparison to their free base forms.

A base addition salt is any organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine.

In some embodiments, the kit further comprises, in addition to the deblocking composition, one or more (e.g., two or more, or three or more) components selected from polymerase, dNTPs (or analogs thereof), fluorescently labeled nucleotides, reversible terminator nucleotides (e.g., dNTPs with a 3′ NO bond), and primers. The kit may further comprise one or more reaction mixtures described below and/or reagents for forming and/or stabilizing a ternary complex. The reaction mixtures and reagents for different steps may be included in separate containers in the kit, such as in an array of tubes accessible by a sequencing system (e.g., by a sipper of a sequencing system, wherein the sipper is configured to deliver the reagents of different tubes to a flow cell comprising target nucleic acids to be sequenced). As such, aspects include a sequencing system comprising the kit comprising the deblocking composition, a flow cell, fluidics for delivering reagents from the kit to the flow cell, and detection optics for sequencing target nucleic acids in the flow cell. Sequencing methods of the subject application may use such a system to perform one or more steps recited herein.

Nucleic acid sequencing reaction mixtures, or simply “reaction mixtures,” can include one or more reagents that are commonly present in polymerase-based nucleic acid synthesis reactions. Reaction mixture reagents include, but are not limited to, enzymes (e.g., polymerase(s)), dNTPs (or analogs thereof), reversible terminator nucleotides, template nucleic acids, primer nucleic acids (e.g., including 3′ blocked primers), salts, buffers, small molecules, agents that remove reversible terminator moieties from reversible terminator nucleotides, co-factors, metals, and ions. The ions may be catalytic ions, divalent catalytic ions, non-catalytic ions that inhibit polymerization, or a combination thereof. The reaction mixture can include salts, such as NaCl, KCl, potassium acetate, ammonium acetate, potassium glutamate, NH₄Cl, or (NH₄HSO₄), that ionize in aqueous solution to yield monovalent cations. The reaction mixture can include a source of ions, such as Mg²⁻ or Mn²⁺, Co²⁺, Cd²⁺ or Ba²⁺ ions. The reaction mixture can include tin, Ca²⁺, Zn²⁺, Cu²⁺, Co²⁺, Fe²⁺ (e.g., Fe(II)SO₄), or Ni²⁺, or other divalent or trivalent non-catalytic metal cations that stabilize ternary complexes by inhibiting formation of phosphodiester bonds between the primed template nucleic acid molecule and the cognate nucleotide. The buffer can include Tris, Tricine, HEPES, MOPS, ACES, IVIES, phosphate-based buffers, and/or acetate-based buffers. The reaction mixture can include chelating agents such as EDTA, EGTA, DTPA, NTA, and the like. Also provided herein are reaction mixtures that can be used during the blocking-and-examination step, as well as reaction mixtures used during removal of ternary complexes and reversible terminator moieties from blocked primers can include one or more of the aforementioned agents. For example, a wash buffer for removing polymerase bound to blocked primed template nucleic acid molecules can include EDTA. A reversible terminator moiety present on 3′-ONH₂ blocked primed template nucleic acid molecules can be removed using a cleavage or deblocking solution including sodium acetate buffer and NaNO₂. Optionally, a single reagent solution is used for both of these purposes.

Optionally, the reaction mixture of a blocking-and-examination step includes a high concentration of salt (e.g., >50 mM); a high pH (e.g., >pH 8.0); 1, 2, 3, 4, or more types of nucleotides; potassium glutamate; a chelating agent; a polymerase inhibitor; a catalytic metal ion; a non-catalytic metal ion; or any combination thereof. The first reaction mixture can include 10 mM to 1.6 M of potassium glutamate (including any amount between 10 mM and 1.6 M). Optionally, the incorporation reaction mixture includes a catalytic metal ion; 1, 2, 3, 4, or more types of nucleotides; potassium chloride; or any combination thereof. In addition to potassium salts, there also can be other salts that provide sources of monovalent cations. Likewise, other glutamate salts also may be useful.

Reagents for forming and/or stabilizing a ternary complex may include a polymerase, a blocked primed template nucleic acid, and a labeled nucleotide (e.g., a labeled non-incorporable nucleotide). For the examination step or sub-step of the provided method, the ternary complex that includes detectably labeled nucleotide (e.g., the labeled non-incorporable nucleotide) is stabilized during the examination step by use of the blocked primed template nucleic acid molecule. Optionally, the examination reaction condition comprises a plurality of blocked primed template nucleic acids, polymerases, labeled nucleotides, or any combination thereof. Optionally, the plurality of labeled nucleotides comprises at least 1, 2, 3, 4, or more types of different non-incorporable nucleotides (e.g., labeled non-incorporable analogs of dATP, dGTP, dCTP, and dTTP or dUTP). Alternatively or additionally, the plurality of nucleotides comprises at most 1, 2, 3, 4, or more types of different non-incorporable nucleotides (e.g., labeled non-incorporable analogs of dATP, dGTP, dCTP, and dTTP or dUTP). Optionally, the plurality of nucleotides includes one or more types of nucleotides that, individually or collectively, complement at least 1, 2, 3 or 4 types of nucleotides in a template (e.g., having the binding specificity of dATP, dGTP, dCTP, and dTTP or dUTP). Optionally, the plurality of template nucleic acids is a clonal population of template nucleic acids.

### III. Methods and Uses of the Disclosure

In another aspect, the present disclosure includes a method of activating a polynucleotide comprising:
contacting the polynucleotide with a deblocking agent, the polynucleotide having an amineoxy group at the base and/or 3′ position to cleave an NO bond of the amineoxy group,
wherein the deblocking agent is a carbonyl phosphonate and/or a carbonyl sulfonate. For example, the amineoxy group may be at the 3′ position.

In another aspect, the present disclosure includes a method of activating a non-extendable polynucleotide comprising:
contacting the non-extendable polynucleotide with a deblocking agent, the non-extendable polynucleotide having an amineoxy group at the base and/or 3′ position to cleave an NO bond of the amineoxy group to obtain an extendable polynucleotide,
wherein the deblocking agent is a carbonyl phosphonate and/or a carbonyl sulfonate. For example, the amineoxy group may be at the 3′ position.

In another aspect, the present disclosure includes a method of extending a non-extendable polynucleotide comprising:
contacting the non-extendable polynucleotide with a deblocking agent, the non-extendable polynucleotide having an amineoxy group at the 3′ position to cleave an NO bond of the amineoxy group to obtain an extendable polynucleotide, and
extending the extendable polynucleotide in a nucleic acid polymerisation,
wherein the deblocking agent is a carbonyl phosphonate and/or a carbonyl sulfonate.

In another aspect, the present disclosure includes a use of a deblocking agent in activating a non-extendable polynucleotide for polymerisation, wherein the non-extendable polynucleotide has an amineoxy group at the 3′ position, and wherein the deblocking agent is as defined in the present disclosure. Extending may also referred to as incorporating or elongating.

In some embodiments, the polynucleotide (e.g., non-extendible polynucleotide) may be a ssDNA, such as a primer hybridized to a template polynucleotide for sequencing with a template-based polymerase or an ssDNA unhybridized at the 3′ end for DNA synthesis by a template-independent polymerase.

In some embodiments, the non-extendable polynucleotide comprises a fluorescent tag and/or a mass tag at the base of the non-extendable polynucleotide, optionally the fluorescent tag and/or the mass tag is reversibly attached to the base.

In some embodiments, the contacting is carried out under conditions suitable to cleave an NO bond between the fluorescent and/or mass tag and the base of the non-extendable polynucleotide.

In some embodiments, the non-extendable polynucleotide is comprised at the 3′ end of a nucleic acid. In some embodiments, the nucleic acid is attached to a surface.

In some embodiments, the polymerization is a template-dependent polymerization, wherein the non-extendable polynucleotide is a primer hybridized to a template polynucleotide.

In some embodiments, the polymerization it a template-independent polymerization by a terminal deoxynucleotidyl transferase.

In some embodiments, the extending is carried out with a non-extendable polynucleotide to obtain a second non-extendable polynucleotide, wherein the non-extendable polynucleotide comprises an amineoxy group at the 3′ position of the nucleotide, and wherein the second non-extendable polynucleotide comprises an amineoxy group at the 3′ position of the second non-extendable polynucleotide.

It can be appreciated that when the extending is carried out with a non-extendable polynucleotide, for example one having a 3′ amineoxy group (e.g. 3′-ONH₂), a non-extendable polynucleotide is obtained having a 3′ amineoxy group. The obtained non-extendable polynucleotide can be subjected to another step of contacting with a deblocking agent to cleave a NO bond in the 3′ amineoxy group, thus forming an extendable polynucleotide, which can be subjected to a further step of extending. Thus, it is contemplated that the methods of the present disclosure can be cyclic. Prior to the contacting of the non-extendable polynucleotide with the deblocking agent, the non-extendable polynucleotide can undergo other steps of interesting including but not limited to detecting, sequencing, chemical modifications (e.g. removal of tags such as fluorescent and/or mass tag and labels), and combinations thereof.

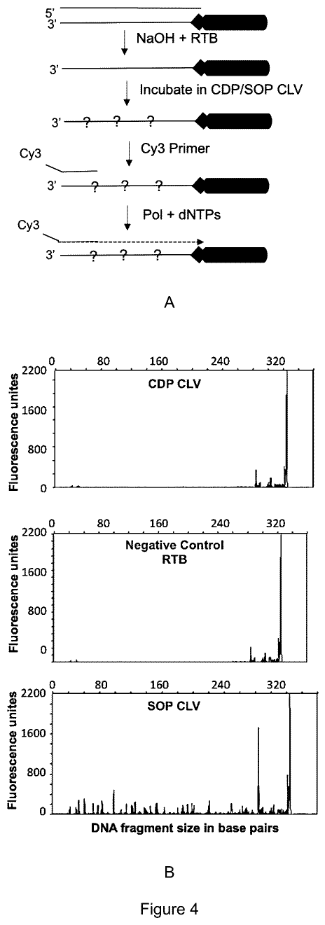

In some embodiments, the method further comprises a plurality of cycles of contacting and extending as defined herein, wherein each extending step forms a further non-extendable polynucleotide that is contacted with the deblocking agent at a later cycle. Between extensions, polymerase may be removed from a polynucleotide (e.g., with a wash solution comprising high salt and/or alcohol) prior to cleaving a 3′ NO bond of the polynucleotide with the deblocking reagent described herein. In other aspects, the polymerase may remain bound to the strand (e.g., primer) it extended during the deblocking step, as the deblocking reagents described herein may be gentler than reagents such as nitrous acid that are commonly used for NO bond cleavage. As demonstrated by FIG. 4, carbonyl diphosphonate show reduced damage to biomolecules such as DNA compared to commonly used cleavage reagents such as nitrous acid. Further, the non-acidic (e.g., pH 6 or above, pH 7 or above, such as pH 6 to pH 9 or pH 7 to pH 8) conditions that carbonyl phosphonate and carbonyl sulfonate can cleave NO bonds at would reduce polymerase damage as compared to acidic conditions (e.g., below pH 6 or below pH 5). Polymerase reuse across cycles may reduce cost of sequencing with a template-based polymerase, or of ssDNA synthesis (e.g., polymerization from an unhybridized 3′ end of an ssDNA) with a template independent polymerase (e.g., TdT). As such, any method described herein may include, or be modified to include, retaining polymerase binding to polynucleotide across multiple cycles of NO bond cleavage at the 3′ end of the polynucleotide.

In some embodiments, after deblocking (e.g., after deblocking in each cycle of a plurality of cycles), the deblocking agent is removed from the polynucleotide. Optionally, polynucleotide may be exposed to a deactivating solution that reacts with or otherwise reduces the activity of the deblocking agent. For example, the deactivating solution may be a basic solution (e.g., having a pH of 8 or greater, or a pH of 9 or greater, or a pH of 10 or greater) and/or may comprise a moiety, such as an small molecule comprising an amineoxy moiety, that reacts with leftover deblocking reagent. This may reduce phasing from unintended deblocking in a next cycle. In certain aspects, the deblocking agent may be flowed to a waste reservoir after a deblocking step, and optionally mixed with the deactivating solution in the waste reservoir (e.g., to reduce reactivity of the waste).

In some embodiments, the method comprises about 3 to about 500 cycles of contacting and extending. For example, the method can comprise at least 3, at least 5, at least 10, at least 15, at least 20, at least 25, at least 35, at least 45, or at least 55 cycles. For example, the method can comprise at most 500, at most 450, at most 400, at most 350, at most 300, at most 250, at most 200, at most 150, at most 100, at most 50, or at most 40 cycles.

In some embodiments, the methods of the present disclosure are high-throughput processes. For example, the method of the present disclosure is a high-throughput sequencing method.

In some embodiments, each of the plurality of cycles further comprises detecting the respective non-extendable polynucleotide obtained in each extending step. As described elsewhere herein, detection may be of the next correct nucleotide, which may be a fluorescent nucleotide incorporated at the 3′ end of the extended polynucleotide (e.g., SBS) or a fluorescent nucleotide stabilized in a ternary complex with the polymerase and the polynucleotide (e.g., SBB). More exotic SBB detection modalities are also suitable, such as where the polymerase is labeled and detected when stabilized in a ternary complex with the next correct nucleotide. In general, any form of imaging fluorescence microscopy may be used to detect fluorescently labeled nucleotides, including but not limited to epifluorescence, confocal, and line scanning microscope setups. Examples of different detection modalities are provided in SBS and SBB references discussed elsewhere herein.

In some embodiments, each of the plurality of cycles further comprises detecting a next cognate nucleotide in a template-based polymerization, wherein the polynucleotide is a primer polynucleotide of a target nucleic acid. The target nucleic acid is a template polynucleotide. Detection may be of single target nucleic acid molecules, or may be of clusters of clonally identically target nucleic acid molecules. For example, a library of target nucleic acid molecules may be ligated or otherwise attached to adapters, and the adapters may provide primer binding site(s) to cluster the target nucleic acids on a solid support (e.g., through bridge amplification or rolling circle amplification). The adapter sequences may also be used for pre-amplification and/or binding of a sequencing primer. The adapter sequences may include components besides primer binding sites, such as sample indices (e.g., barcode subsequences) and molecular indices (e.g., randomer subsequences). Examples of clustering and sequencing workflows are provide in US Patent Publication No. 2022/0349002, which is incorporated herein by reference.

In some embodiments, the next cognate nucleotide is a fluorescently labeled nucleotide that is bound in a ternary complex but is not incorporated into the polynucleotide, wherein the ternary complex comprises a polymerase, the polynucleotide primer and template polynucleotide, and the fluorescently labeled nucleotide. In some embodiments, the next cognate nucleotide is a fluorescently labeled nucleotide that is incorporated into the polynucleotide.

In some embodiments, the method is part of a method for determining a sequence of the template polynucleotide, e.g., from detecting the next cognate nucleotide in each of the plurality of cycles.

In some embodiments, a method of cyclic polynucleotide extension with a polymerase may include a) incorporating, by a polymerase, a non-extendable nucleotide at the 3′ end of a polynucleotide, wherein the non-extendable nucleotide comprises an amineoxy bond; and b) cleaving the amineoxy bond with a carbonyl phosphonate or a carbonyl sulfonate. The incorporation and/or cleavage may be according to any of the embodiments described herein. For example, cleaving may be with a carbonyl diphosphonate, optionally in the presence of an amine counterion. The method may further include cycles of step a) and b), optionally wherein the polymerase remains bound to the polynucleotide across the cycles of step a) and b). The cycles may further include detecting a fluorescent nucleotide bound by the polymerase before or after each step a) of incorporating the non-extendible nucleotide, optionally wherein the fluorescent nucleotide is in a stabilized ternary complex with the polymerase and the polynucleotide.

In some embodiments, a method of extending a non-extendable polynucleotide includes: contacting the non-extendable polynucleotide with a deblocking solution comprising a deblocking agent and a solubility enhancer, the non-extendable polynucleotide having an amineoxy group at the 3′ position, to cleave an NO bond of the amineoxy group to obtain an extendable polynucleotide, and extending the extendable polynucleotide in a nucleic acid polymerization in which a polymerase incorporates a nucleotide having an amineoxy group at the 3′ position, and repeating the steps of contacting and extending. The contacting and/or extending may be according to any of the embodiments described herein. For example, the deblocking agent may a carbonyl diphosphonate. The solubility enhancer may be an amine that acts as a counterion to the deblocking agent. The polymerase is a template-based polymerase, such as in a DNA sequencing reaction, or a template-independent polymerase, such as in a DNA synthesis reaction.

In another aspect, the present disclosure includes methods of sequencing using the cleavage chemistry described herein. The sequencing may be a cyclical sequencing reaction, such as sequencing by binding (SBB), as described elsewhere herein, or sequencing-by-synthesis (SBS). Sequencing may be of a target nucleic acid bound to a surface (e.g., a bead, hydrogel, flow cell surface, and the like). Identical “clusters” of a target nucleic acid may provide signal amplification, although single molecule sequencing of target nucleic acids are also suitable. Clusters may be formed in emulsion, by bridge amplification, or by rolling circle amplification.

Sequencing by synthesis (SBS) generally involves the enzymatic extension of a nascent primer through the iterative addition of labeled nucleotides against a template strand to which the primer is hybridized. After labeled nucleotides are detected, a cleavage step may remove the label (e.g., a fluorophore coupled, such as through an NO bond, to the base of the nucleotide) and blocker (e.g., an NO bond at the 3′ end of the nucleotide) to allow for the next cycle of extension, detection and cleavage. Briefly, SBS can be initiated by contacting target nucleic acids, which may be attached to a surface in a flow cell, with one or more labeled nucleotides, DNA polymerase, etc. A primer extended using the target nucleic acid as a template will incorporate a labeled nucleotide that can be detected. Optionally, the labeled nucleotides can further include a reversible termination moiety, such as the amineoxy group as described herein, that terminates further primer extension once a nucleotide has been added to a primer. For example, a nucleotide analog having a reversible terminator moiety can be added to a primer such that subsequent extension cannot occur until a deblocking agent is delivered to remove the moiety. Thus, for embodiments that use reversible termination, a deblocking agent can be delivered to the flow cell (before or after detection occurs). Washes can be carried out between the various delivery steps. The cycle can then be repeated n times to extend the primer by n nucleotides, thereby detecting a sequence of length n. Exemplary SBS procedures, reagents and detection instruments that can be readily adapted for use with an array produced by the methods of the present disclosure are described, for example, in Bentley et al., Nature 456:53-59 (2008), WO 04/018497; WO 91/06678; WO 07/123744; U.S. Pat. Nos. 7,057,026; 7,329,492; 7,211,414; 7,315,019 or 7,405,281, and US Pat. App. Pub. No. 2008/0108082 A1, each of which is incorporated herein by reference. Also useful are SBS methods that are commercially available from Illumina, Inc., San Diego Calif.

Embodiments may include NO cleavage and nucleotide extension in cycles of a Sequencing by Binding (SBB) method. Examples of SBB are described in US Patent Pub. Nos. 2018/0208983, 2020/0032317 and 2021/0340612, which is incorporated by reference herein. In comparison to SBS methods that detect an incorporated nucleotide, SBB methods detect an unincorporated nucleotide stabilized in a ternary complex (i.e., stabilized with polymerase and primer-template). SBB procedures are typically carried out as a series of cycles, with each cycle including one or more steps that result in identification of the next correct nucleotide for a particular nucleotide position of a primed template nucleic acid. As such, the sequence of the nucleic acid template is determined from the series of nucleotides identified in the series of cycles. Convenient platforms for the sequencing chemistry can involve flow cells or individual wells of a multiwell plate, where the different nucleic acids may be present as features such as in vitro- or in situ-synthesized clusters of primed template nucleic acids, or such as immobilized microbeads displaying primed template nucleic acid molecules. Cognate nucleotide identification can be made by identifying label associated with the nucleotide analog used in the procedure. This can be carried out using as few as a single imaging step to detect each of four different types of cognate nucleotide (i.e., labeled non-incorporable nucleotide analogs of: dATP, dGTP, dCTP, and dTTP or dUTP).

Generally speaking, the polymerase used in nucleic acid sequencing by binding reactions undergoes transitions between open and closed conformations during discrete steps of the reaction. In one step, the polymerase binds to a primed template nucleic acid to form a binary complex, also referred to herein as the pre-insertion conformation. In a subsequent step, an incoming nucleotide is bound and the polymerase fingers close, forming a pre-chemistry conformation comprising the polymerase, primed template nucleic acid and nucleotide (i.e., a ternary complex); wherein the bound nucleotide has not been incorporated. The two steps can either use the same polymerase, or different polymerases.

A catalytic metal ion (e.g., Mg²⁺ or Mn²⁺) catalyzing chemical incorporation of the next correct nucleotide (e.g., a cognate incorporable reversible terminator nucleotide) can promote phosphodiester bond formation if the primer strand is free of any reversible terminator moiety. Here, nucleophilic displacement of a pyrophosphate (PPi) by the 3′-hydroxyl of the primer results in phosphodiester bond formation. This is generally referred to as nucleotide “incorporation.” The polymerase returns to an open state upon the release of PPi following nucleotide incorporation, and translocation initiates the next round of reaction. Certain details of Sequencing By Binding™ procedures can be found in commonly owned U.S. patent applications identified by Ser. No. 14/805,381 (published as U.S. 2017/0022553 A1) and 62/375,379, the entire disclosures of these documents being incorporated by reference herein for all purposes.

While a ternary complex including a primed template nucleic acid molecule can form in the absence of a divalent catalytic metal ion (e.g., Mg²⁺ or Mn²⁺), chemical addition of nucleotide can proceed in the presence of the divalent metal ions if the primed template nucleic acid molecule is free of any reversible terminator moiety. Low or deficient levels of catalytic metal ions, such as Mg²⁺ tend to lead to non-covalent (physical) sequestration of the next correct nucleotide in a tight ternary complex. Those having an ordinary level of skill in the art will appreciate that non-catalytic metal ions that inhibit incorporation also can be used for stabilizing ternary complexes. Certain procedures detailed below that employ a step for forming ternary complexes containing reversible terminator nucleotides in the absence of labeled nucleotides can use non-catalytic metal ions to stabilize ternary complexes preliminary to carrying out the blocking-and-examination step. Again, incorporation of the reversible terminator and detection of labeled nucleotide interaction with the blocked primer in a ternary complex can take place in the same reaction mixture during this combined blocking-and-examination step.

A blocked primer terminating at its 3′-end with a reversible terminator nucleotide that precludes phosphodiester bond formation also can be used for stabilizing ternary complex formation. The product of a blocking-and-examination step includes blocked primers that stabilize ternary complexes. In any reaction step described above, formation of a stabilized ternary complex containing a detectably labeled nucleotide that is not incorporated may be monitored to identify the next correct base in the nucleic acid sequence. Reaction conditions can be changed to disengage the polymerase and labeled cognate nucleotide from a blocked primed template nucleic acid molecule, and changed again to remove from the local environment any reversible terminator moiety attached to the nucleotide at the 3′-end of the primer strand of the primed template nucleic acid molecule. In some embodiments, both the polymerase and cognate nucleotide of the ternary complex, and the reversible terminator moiety are removed in a single step using a reagent that dissociates ternary complexes and cleaves the reversible terminator moiety from its position at the 3′-end of the blocked primed template nucleic acid molecule.

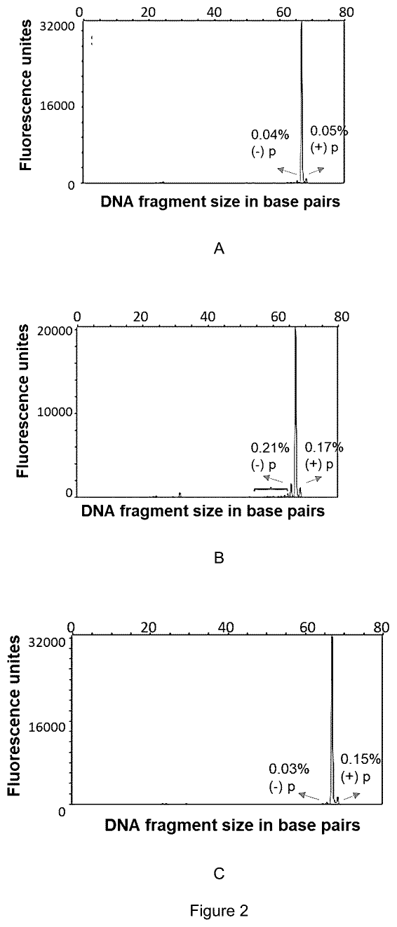

Generally speaking, the disclosed Sequencing By Binding procedure includes an “examination” step or sub-step that detects signals useful for identifying the next template base. The detected signals reflect the dynamic equilibrium interaction of detectable label with primed template nucleic acid molecules of a nucleic acid feature. Optionally, the detected label is covalently linked to the cognate nucleotide. Optionally, the linkage joins the label to the base moiety of the nucleotide. Exemplary linkers are shown in FIG. 2 and in the Formulas below. Optionally, the label attached to the base is the only label attached to the nucleotide, meaning that neither the pentose sugar nor any phosphate moiety of the nucleotide is attached to a detectable label (e.g., an exogenous fluorescent label). Identity of the cognate nucleotide downstream of reversibly blocked primer is determined from signals detected without linking the cognate nucleotide to the 3′-end of the primer by a covalent bond.

It can be appreciated that in some instances, the cleavage of the NO bond may not be complete, such that the contacting step does not produce an extendable polynucleotide, or that a portion of the non-extendable polynucleotide remains non-extendable after the contacting step. Such non-extendable polynucleotide cannot participate in the extending step. As such, it is contemplated that some of the plurality of cycles of contacting and extending can be interspersed with cycles where the contacting does not cleave an NO bond of the amineoxy group of all or a portion of the non-extendable polynucleotide and where the extending step does not take place. In some embodiments, the efficiency of the cleaving of the NO bond of the amineoxy group in the contacting step is at least 30%, at least 40%, at least 50%, at least 60%, at least 70%, at least 80%, at least 90%, at least 95%, at least 97%, or at least 99%. In some embodiments, the efficiency of the cleaving is about 100%.

Some embodiments include methods of DNA synthesis, such as chemical or enzymatic DNA synthesis. Enzymatic DNA synthesis may be with a template-independent polymerase, such as Terminal deoxynucleotidyl transferase (TdT), such as is described in US Patent Publication Nos. 2019/0300923 and 2019/0112627.

In some embodiments, synthesis of a ssDNA nucleic acid of a desired sequence may include: elongating initial nucleic acid fragments or attached to a support and having free 3′-hydroxyls by: (a) reacting said fragments with a modified nucleotide and a template-independent DNA polymerase, wherein said modified nucleoside triphosphate comprises a 3′ NO group that prevents multiple additions of nucleotides, (b) cleaving the NO group with a deblocking reagent or solution described herein, and (c) repeating steps (a) and (b).

In some embodiments, synthesis of an ssDNA nucleic acid of a desired sequence may include elongating initial nucleic acid fragments or attached to a support and having free 3′-hydroxyls by: (a) reacting said fragments with a modified nucleoside triphosphate and a template-independent DNA polymerase, wherein said modified nucleotide comprises a linker comprising an NO group is joined to the base of the nucleotide and coupling the nucleotide to the polymerase, (b) cleaving the NO group with a deblocking reagent or solution described herein, and (c) repeating steps (a) and (b). The coupling of the nucleotide to the polymerase acts as a reversable termination, as further elongation may not be possible until after cleavage of the linker. The linker comprising an NO group is joined to the base of the nucleotide, such as at an atom of the base that is not involved in base pairing. In other embodiments, the linker is attached to an aldehyde specifically generated within the polymerase, as described in Carrico et al. (Nat. Chem. Biol. 3, (2007) 321-322). This aldehyde may then be specifically labeled with the aminooxy moiety of a linker. In some embodiments, the linker may be attached to the polymerase via non-covalent binding of a moiety of the linker to a moiety fused to the polymerase. Examples of such attachment strategies include fusing a polymerase to streptavidin that can bind a biotin moiety of a linker, or fusing a polymerase to anti-digoxigenin that can bind a digoxigenin moiety of a linker.

As described in the above embodiments, the amineoxy group cleaved by the deblocking agent may be on the 3′ end and/or base of a nucleotide. As such, any of the kits, methods and systems described herein with a 3′ amineoxy group may, alternatively or in addition to the 3′ amineoxy group, have an amineoxy group off the base.

### EXAMPLES

The following non-limiting examples are illustrative of the present disclosure.

### General Methods

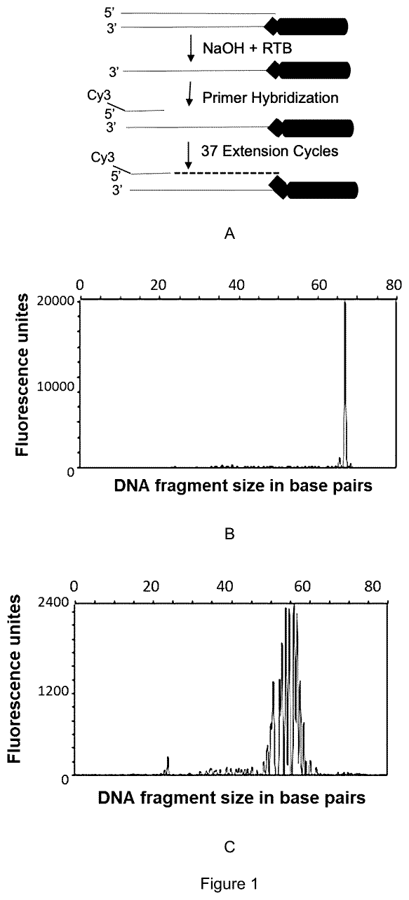

Polynucleotides were extended in an Octet™ system as shown in FIG. 1A. The polynucleotides were Cy3-primers hybridized to biotinylated template DNA which were in turn bound to streptavidin coated tips of the Octet system. The Octet system move a set of 8 tips between the wells of a heated 96-well plate, for a set number of SBB extension cycles. Each cycle included an IMG step under conditions at which fluorescent nucleotides of ternary complexes could be imaged, an RTN step in which amineoxy terminated nucleotides were incorporated at the 3′ end of the primer by a polymerase, a wash step that removes excess nucleotides, and a CLV step that exposes the amineoxy terminated extended primer to a deblocking agent or candidate. The length of extended polynucleotides were then detected by capillary electrophoresis (CE), as described in US Patent Pub. No. 2019/0241945, the content of which is incorporated herein.

A library of polynucleotides were sequenced in a sequencing-by-binding workflow, and On/Off signal intensity was measured in each sequencing cycle, as described in US Patent Pub. No. 2020/0032317, the content of which is incorporate herein. Specifically, ‘on’ signal intensity (corresponds to the binding of the cognate nucleotide) and ‘off’ signal intensity (corresponds to the binding of the non-cognate nucleotide) in each SBB cycle. By using [NaNO2] 2 M and considering pH 5.2 and based on Henderson-Hasselbalch equation, we calculated that 18 mM of the nitrous acid exists in CLV (Cleave) reagent. Although, this cleave formulation is efficient in converting dNTP-RT to dNTP on the sequencing strand, however, it may have one or more disadvantages, such as instability, corrosivity, and/or DNA damage. Regarding instability: nitrous acid in our current CLV reagent degrades and produces nitrogen oxide gas (NOx) which weaken strength of the deblocking reagent. Nitrous acid is very corrosive and has material compatibility with plastics consumables and instruments. Current cleave electropherograms (CE data) from DNA damage Run-Off Assays show that nitrous acid is damaging to the DNA.

Unless described otherwise, NaNO₂ is used at pH 5.2 and CDP refers to a carbonyl diphosphonate having just one carbon atom

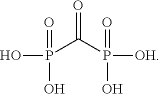

### Example 1 Cycles of Extension Using Octet/CE Assay

Polynucleotide sequence was synthesized using the Octet/CE method where non-extendable polynucleotides having a 3′ amineoxy group were used to reversibly stop extension, as shown in the workflow scheme of FIG. 1A. The 3′amineoxy group was cleaved using the standard NaNO₂ at pH 5.2 during 37 cycles of extension, as shown in FIG. 1B. After 24 hours of exposure to the standard cleavage condition, the extended polynucleotide showed degradation (FIG. 1C). Compared to the standard operating procedure (SOP) using sodium nitrate, CDP as a deblocking agent provided must cleaner product having minimal dephasing and no DNA damage.

Despite producing stable clean product, when used alone CDP showed higher positive dephasing and higher negative dephasing on electropherogram compared by SOP sodium nitrate cleave in sodium acetate buffer. (See FIG. 2 panels A and B) CDP further exhibited slight solubility issue under some conditions. The reaction condition for CDP was then optimized by using a buffer and an amine solubility enhancer. The optimized CDP composition comprising sodium acetate buffer at pH about 5.2 to about 5.6 and N,N′-Dimethylethylenediamine as a solubility enhancer showed slightly higher but acceptable positive dephasing but lower negative dephasing and no solubility issues. (FIG. 2C) The optimization of the buffer and the solubility enhancer is shown in Example 2.

### Example 2 Optimization of Buffer, pH and Solubility Enhancer

CDP was formulated with a number of different buffers at different pH and various different amines as solubility enhancers. 47 formulations were made and tested. In general, CDP was dissolved in water and loaded onto Dowex 50™ cation exchange resin, and incubated for a few minutes. The resin was eluted with an aqueous solution of the amine solubility enhancer. The resulting solution was either used directly in Octet/CE assays or dried form a solid that was very soluble in water. The buffer candidates and solubility enhancers tried are presented in Tables 1A and B. FIG. 2 shows electropherograms of Octet/CE cyclic extension assays using 37 cycles of extension with either NaNO₂ (Panel A), CDP alone (Panel B) or CDP and N,N′-Dimethylethylenediamine in sodium acetate buffer (Panel C) as deblocking composition. The NaNO₂ deblocking composition showed low phasing (0.04% negative phasing and 0.05% positive phasing), while the CDP alone condition showed higher phasing (0.21% negative phasing and 0.17% positive phasing). The addition of an amine counterion, which may act as a solubility enhancer, to the CDP deblocking composition reduced the negative phasing to 0.03%.

TABLE 1A
Buffers and pH

| Buffer | pH |
|---|---|
| Sodium Acetate | 5.2-5.6 |
| Potassium phosphate | 7.0 |
| HEPES | 7.5-8.2 |
| Tricine | 7.0-8.2 |

TABLE 1B
Solubility Enhancer
Solubility Enhancer

N,N′-Dimethylethylenediamine
N,N′-Diethylethylenediamine
1-Methylimidazole
2-Amino-5-methoxybenzoic acid
2-(Aminomethyl)benzimidazole
Triethylamine
DABCO
DBU

While all buffers, pH and solubility enhancer combinations worked, sodium acetate and N,N′-Dimethylethylenediamine provided the best performance.

### Example 3 Sequencing: On/Off by Cycle

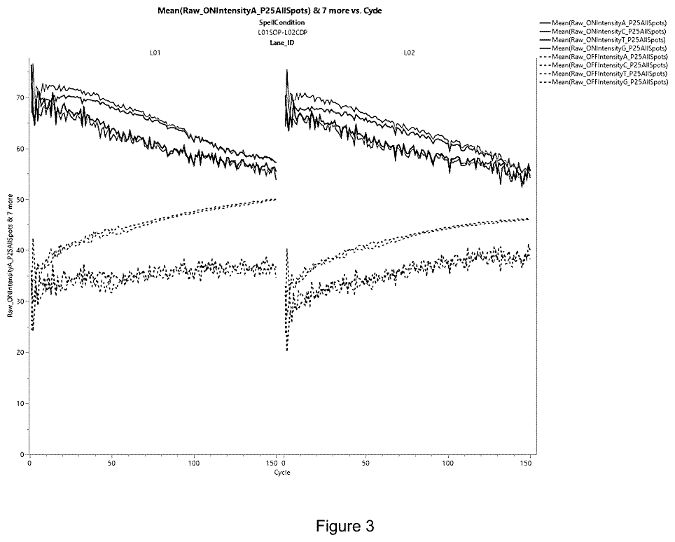

FIG. 3 shows raw intensities of on/off sequencing run. Y axis is on/off intensities. Each solid line of different color is signal (ON) from a different base, and each dotted line is background for that signal (OFF) for a different base. 150 cycles total. A library of polynucleotides were sequenced in a sequencing-by-binding workflow, and On/Off signal intensity was measured in each sequencing cycle, as described in US Patent Pub. No. 2020/0032317, the content of which is incorporate herein. Specifically, ‘on’ signal intensity (corresponds to the binding of the cognate nucleotide) and ‘off’ signal intensity (corresponds to the binding of the non-cognate nucleotide) in each SBB cycle.

The raw intensities data are shown in FIG. 3. After 150 cycles, the signal to noise ratio was comparable between CDP and SOP (sodium NO₂), indicating that CDP can be used effective blocking agent for prolonged sequencing runs.

### Example 4

FIG. 4 shows electropherograms of Octet/CE runoff assay design (Panel A) and results (Panel B) showing reduction in ssDNA damage by CDP compared to NaNO₂. Octet tips were bound to Cy3-primer template as described for FIG. 1A, and then contacted with an extension mixture of polymerase and dNTP to allow for continued extension. As shown in FIG. 4B top panel, pre-incubation with CDP cleave mixture did not interfere with extension as the extension product CE trace is similar to extension in the absence of a cleave reagent (FIG. 4B middle panel). However, consistent with the degradation shown in FIG. 4C, preincubation with NaNO₂ cleave mixture of FIG. 1B and FIG. 1C interfered with extension, likely through damage to the template. As shown in FIG. 3 and FIG. 4, CDP has cleavage comparable to NaNO₂ (low phasing) without significant damage to the DNA template.

### Example 5

A number of other carbonyl compounds were test for their ability to cleave the NO bond at the 3′ position of a polynucleotide, according to the assay design shown in FIG. 1A. The results are listed in the Table 4.

TABLE 2
Different Deblocking Agents

| | | | Extension at 52 C. |
|---|---|---|---|
| | | Extension at 40 C. in 37 | in 37 |
| | Deblocking Candidate | cycles Octet CE assay | cycles octet/Ce assay |
| 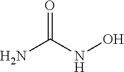 | Hydroxycarbamide | No extension | |
| 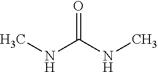 | Dimethyl urea | No Extension | |
| 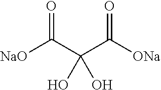 | sodium mesoxalate | No Extension | |
| 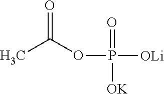 | LiK Aceyl Phosphate | No Extension | |
| 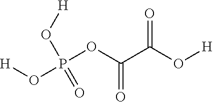 | 2-Hydroxy-2-phosphonooxyacetic acid | No Extension | |
| 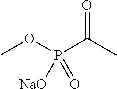 | methyl acetylphosphonate CH3—CO—PO(Ome)—O⁻ Na⁺ | No Extension | |
| 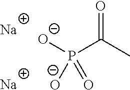 | Di sodium acetylphosphonate | 12 bp Extension | 3 bp Extension |
| 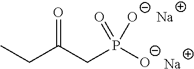 | Di sodium (2-oxobutyl)phosphonate | 4 bp Extension | 4 bp Extension |
| 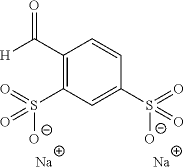 [SMILES: O=Cc1ccc(S(=O)(=O)[O-])cc1S(=O)(=O)[O-] ; [Na+] ; [Na+]] | Di sodium 4-formylbenzene-1,3-disolfonate | 3 bp Extension | 3 bp Extension |
| 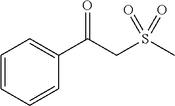 | Methyl sulfonyl acetophenone | 5 bp Extension | 5 bp Extension |
| 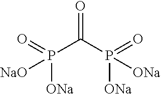 | Carbonyl diphosphonate (CDP) | 37 bp Extension | |

As shown in Table 4, some types of carbonyl compounds including urea, mesoxalate, and phosphate compounds failed to cleave NO bond. A number of deblocking candidates, including carbonyl phosphonates and carbonyl sulfonates, were successful in cleaving NO bond in multiple cycles, allowing for multiple base extension. Compound showing deblocking (e.g., extension in multiple cycles) in Table 4 include di sodium acetylphosphonate, di sodium (2-oxobutyl)phosphonate, di sodium 4-formylbenezene-1,3-disolfonate, methyl sulfonyl acetophenone, and carbonyl diphosphonate (CDP). These compounds include certain ketones and aldehydes, certain cyclic and aromatic structures, compounds comprising a single phosphonate or a single sulfonate, and compounds in which a phosphonate or sulfonate is spaced from the oxygen of a ketone or aldehyde by up to three carbons. Deblocking candidates were tested at various concentrations, generally between 1 and 100 mM (e.g., at 1 mM, 2 mM, 4 mM, 8 mM, 20 mM, 35 mM, 40 mM, 70 mM, and/or 100 mM for different candidates), in acetate (1 M) and tris (0.2 M) buffers, and buffered at pH 6 or 8, with and without amine counterion. Across these conditions, the carbonyl phosphonates and carbonyl sulfonates demonstrated cleavage of NO bonds across multiple cycles. Candidates that were tested at both 40 degrees Celsius and 52 degrees Celsius demonstrated cleavage of NO bonds across multiple cycles at both temperatures.

While the present disclosure has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term.

1.-64. (canceled)

65. A method of extending a non-extendable polynucleotide comprising
contacting the non-extendable polynucleotide with a deblocking agent, the non-extendable polynucleotide having an amineoxy group at the 3′ position, to cleave an NO bond of the amineoxy group to obtain an extendable polynucleotide, and
extending the extendable polynucleotide in a nucleic acid polymerization,
wherein the deblocking agent is a carbonyl diphosphonate.

66. The method of claim 65, wherein the polymerization is a template-dependent polymerization, wherein the non-extendable polynucleotide is a primer hybridized to a template polynucleotide.

67. The method of claim 65, wherein the polymerization is a template-independent polymerization by a terminal deoxynucleotidyl transferase.

68. The method of claim 65, wherein the extending is carried out with a non-extendable polynucleotide to obtain a second non-extendable polynucleotide, wherein the non-extendable polynucleotide comprises an amineoxy group at the 3′ position of the nucleotide, and wherein the second non-extendable polynucleotide comprises an amineoxy group at the 3′ position of the second non-extendable polynucleotide.

69. The method of claim 65, wherein the method further comprises a plurality of cycles of contacting and extending, wherein each extending step forms a further non-extendable polynucleotide that is contacted with the deblocking agent at a later cycle.

70. The method of claim 69, wherein each of the plurality of cycles further comprises detecting the respective non-extendable polynucleotide obtained in each extending step.

71. The method of claim 70, wherein each of the plurality of cycles further comprises detecting a next cognate nucleotide in a template-based polymerization, wherein the polynucleotide is a primer of a template polynucleotide.

72. The method of claim 71, wherein the next cognate nucleotide is a fluorescently labeled nucleotide that is bound in a ternary complex but is not incorporated into the polynucleotide, wherein the ternary complex comprises a polymerase, the polynucleotide primer and template polynucleotide, and the fluorescently labeled nucleotide.

73. The method of claim 72, wherein the next cognate nucleotide is a fluorescently labeled nucleotide that is incorporated into the polynucleotide.

74. The method of claim 65, wherein the non-extendable polynucleotide comprises a fluorescent tag at the base of the non-extendable polynucleotide, wherein the fluorescent tag is reversibly attached to the base, and wherein the contacting further comprises cleaving an NO bond between the fluorescent the base of the non-extendable polynucleotide.

75. The method of claim 65, wherein extending is by a polymerase that incorporates a nucleotide having an amineoxy group at the 3′ position.

76. The method of claim 65, wherein the deblocking agent is comprised in a deblocking composition, the deblocking composition comprising the deblocking agent and a buffer.

77. The method of claim 76, wherein the deblocking composition has a pH of 4 to 7.

78. The method of claim 77, wherein the deblocking composition has a pH of 4.5 to 6.

79. The method of claim 76, wherein the buffer comprises or is acetate.

80. The method of claim 76, wherein the deblocking solution further comprises a solubility enhancer.

81. The method of claim 80, wherein the solubility enhancer is an amine.

82. The method of claim 81, wherein the amine is selected from substituted or unsubstituted nitrogen-containing heteroaromatic, substituted or unsubstituted arylamine, alkylamine, substituted or unsubstituted nitrogen-containing heterocycle, and combinations thereof.

83. The method of claim 82, wherein the amine is N,N′-dimethylethylenediamine.

84. The method of claim 83, wherein the solubility enhancer is present in the deblocking composition at a concentration of about 10 mM to about 50 mM.

85. The method of claim 65, wherein a carbonyl of the deblocking agent is an aldehyde.

86. The method of claim 65, wherein a carbonyl of the deblocking agent is a keytone.

87. The method of claim 65, wherein the deblocking agent is 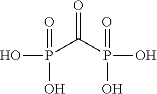
or a salt thereof.

88. A method of cyclic polynucleotide extension with a polymerase, comprising:
a) incorporating, by a polymerase, a non-extendable nucleotide at the 3′ end of a polynucleotide, wherein the non-extendable nucleotide comprises an NO bond; and
b) cleaving the NO bond, wherein cleaving is with a carbonyl phosphonate; and
c) repeating steps a) and b) in a plurality of cycles.

89. The method of claim 88, wherein the NO bond is of an amineoxy group.

90. The method of claim 88, wherein the carbonyl phosphonate is a carbonyl diphosphonate.

91. The method of claim 88, wherein the carbonyl diphosphonate is in the presence of an amine counterion.

92. The method of claim 88, wherein the polymerase remains bound to the polynucleotide across the cycles of step a) and b).

93. The method of claim 88, further comprising detecting a fluorescent nucleotide bound by the polymerase before or after each step a) of incorporating the non-extendible nucleotide, wherein the fluorescent nucleotide is in a stabilized ternary complex with the polymerase and the polynucleotide.

94. The method of claim 88, wherein the polymerization is a template-independent polymerization by a terminal deoxynucleotidyl transferase.
